(19) United States
(12) Patent Application Publication
(10) Pub. No.: US 2023/0165856 A1
(43) Pub. Date: Jun. 1, 2023

(54) DAPAGLIFLOZIN AND AMBRISENTAN FOR THE PREVENTION AND TREATMENT OF COVID-19

(71) Applicant: AstraZeneca AB, Södertälje (SE)

(72) Inventors: Philip AMBERY, Södertälje (SE); Peter GREASLEY, Södertälje (SE); Sven MOOSMANG, Södertälje (SE); Magnus ALTHAGE, Södertälje (SE)

(21) Appl. No.: 17/997,372

(22) PCT Filed: Apr. 28, 2021
(86) PCT No.: PCT/EP2021/061067

§ 371 Date: Oct. 28, 2022

Related U.S. Application Data
(60) Provisional application No. 63/017,317, filed on Apr. 29, 2020.

(51) Int. Cl. A61K 31/505; A61K 31/70; A61P 31/14

(52) U.S. Cl. CPC A61K 31/505; A61K 31/70; A61P 31/14

(57) ABSTRACT

The present disclosure is directed to the use of dapagliflozin and ambrisentan for the treatment and prevention of coronavirus disease 2019 (COVID-19), SARS-CoV-2 infection, and/or symptoms thereof.

### BACKGROUND

Human coronaviruses (CoVs) are single-stranded positive-sense RNA viruses. Over the past two decades, zoonotic transmission events have led to the emergence of two highly pathogenic CoVs: severe acute respiratory syndrome (SARS)-CoV and Middle East respiratory syndrome (MERS)-CoV. SARS-CoV-2, which causes the disease known as COVID-19, was first reported in late 2019 and was declared a pandemic on 11 Mar. 2020 by World Health Organisation (WHO). The virus's mortality rate is currently uncertain, but the virus has a greater impact on patients of advanced age, with type 2 diabetes, cardiac disease, chronic obstructive pulmonary disease (COPD), and/or obesity. Recent information on patients at risk for developing serious complications, including death, in the setting of COVID-19, indicate that those with cardiometabolic disease (obesity, hypertension, type 2 diabetes, atherosclerotic cardiovascular disease, heart failure, and/or kidney disease at baseline) are at much greater risk (JAMA. 2020; 323(16):1612-1614. doi:10.1001/jama.2020.4326, JAMA. 2020; 323(16):1545-1546. doi:10.1001/jama.2020.4031).

Most patient contracting the virus have mild symptoms, but in some patients, the infection in the lung is severe causing severe respiratory distress or even death. COVID-19-related complications (CRC) include acute respiratory distress syndrome (ARDS), arrhythmia, shock, acute kidney injury, acute cardiac injury, liver dysfunction and secondary infection. There is currently no therapeutic treatment currently available for COVID-19.

Dapagliflozin is a potent, highly selective, and orally active inhibitor of human renal sodium-dependent glucose transporter 2 (SGLT2) (SGLT2i) that has been approved to improve glycemic control in adults with type 2 diabetes mellitus (as an adjunct to diet and exercise) and to reduce the risk of hospitalization for heart failure in adults with type 2 diabetes mellitus and established cardiovascular disease or multiple cardiovascular risk factors.

The chemical structure of dapagliflozin is:

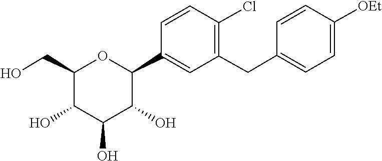 [SMILES: CCOc1ccc(Cc2cc([C@@H]3O[C@H](CO)[C@@H](O)[C@H](O)[C@H]3O)ccc2Cl)cc1]

Ambrisentan is a potent orally active endothelin-1 (ET) receptor antagonist (ERA) selective for the endothelin A (ETA) receptor. Endothelin-1 plays a significant role in the pathophysiology of Pulmonary Arterial Hypertension (PAH). It is approximately 4000-fold more selective for the ETA versus the ETB receptor. It is licensed for use in PAH at a dose of 5 mg and up to 10 mg.

The chemical structure of ambrisentan is:

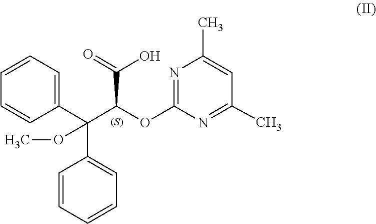

There is a need for an immediate response capable of reducing the mortality rate of COVID-19 in vulnerable patients.

### SUMMARY OF THE DISCLOSURE

Provided herein are methods of treating and/or preventing coronavirus disease 2019 (COVID-19) in a patient comprising administering to the patient a pharmaceutical composition comprising a combination of an effective amount of a compound of formula (I)

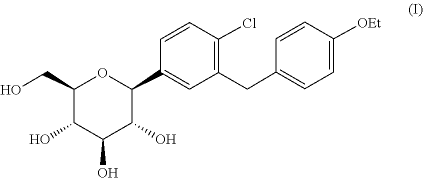 [SMILES: CCOc1ccc(Cc2cc([C@@H]3O[C@H](CO)[C@@H](O)[C@H](O)[C@H]3O)ccc2Cl)cc1]

or prodrugs thereof and a pharmaceutical composition comprising an effective amount of a compound of formula (II)

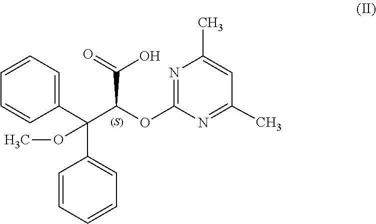

or prodrugs thereof.

Also provided herein are methods of treating or preventing renal insufficiency induced by severe acute respiratory syndrome coronavirus 2 (SARS-CoV-2) infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

Also provided herein are methods of treating or preventing cardiovascular insufficiency induced by SARS-CoV-2 infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

Also provided herein are methods of treating or preventing respiratory insufficiency induced by SARS-CoV-2 infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

Also provided herein are methods of treating or preventing worsening respiratory insufficiency induced by SARS-CoV-2 infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

Also provided herein are methods of treating and/or preventing a cytokine storm in a patient infected by SARS-CoV-2 or a patient with COVID-19, the method comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

Also provided herein are methods of reducing the rate of a composite endpoint of death, worsening respiratory insufficiency, or acute insufficiency in renal and/or cardiovascular systems in a patient with COVID-19, comprising administering to the patient a combination of a pharmaceutical composition comprising an effective amount of a compound of formula (I) or prodrugs thereof and a pharmaceutical composition comprising an effective amount of a compound of formula (II) or prodrugs thereof, wherein the rate of the composite endpoint is reduced relative to an administration regimen where the patient does not receive a combination of a pharmaceutical composition comprising an effective amount of a compound of formula (I) or prodrugs thereof and a pharmaceutical composition comprising an effective amount of a compound of formula (II) or prodrugs thereof.

In some aspects provided herein, the patient has confirmed or suspected SARS-CoV-2 infection requiring hospitalization.

In some aspects provided herein, the patient has a history of any one or more of the following conditions: heart failure, including heart failure with reduced ejection fraction (HFrEF) and heart failure with preserved ejection fraction (HFpEF); myocardial infarction, including acute and chronic; coronary artery disease; stroke, including ischemic, hemorrhagic, or undetermined; hypertension; obesity; kidney disease such as chronic kidney disease, such as stage 3 chronic kidney disease (eGFR≥30 mL/min/1.73 m2); and/or Type 2 diabetes.

In some aspects provided herein, the patient has respiratory insufficiency.

In some aspects provided herein, the patient has an O₂ saturation of ≤94% without additional oxygen.

In some aspects provided herein, the patient has an O₂ saturation of ≥94%

In some aspects provided herein, the patient requires mechanical ventilation or oxygen.

In some aspects provided herein, the patient does not require mechanical ventilation or oxygen.

In some aspects provided herein, the patient has a respiratory rate of greater than or equal to 24 breaths/minute.

In some aspects provided herein, the patient has an O₂ saturation of >94% and respiratory rate <24 breaths/minute without supplemental oxygen.

In some aspects provided herein, the patient has pneumonia.

In some aspects provided herein, the patient is not mechanically ventilated via endotracheal intubation.

In some aspects provided herein, the patient does not have insufficiency in an organ system other than the renal and cardiovascular organ systems.

In some aspects provided herein, the patient does not have type 2 diabetes mellitus (T2DM.) In some aspects provided herein, the patient has T2DM.

In some aspects provided herein, the patient is at least 40 years of age, at least 50 years of age, at least 60 years of age, at least 70 years of age, at least 80 years old, or at least 90 years old.

In at least one embodiment, the compound of formula (I) is in the form of a pharmaceutically acceptable solvate, mixed solvate, or complex. In some aspects provided herein, the compound of formula (I) is in the form of a non-crystalline solid. In some aspects provided herein, the compound of formula (I) is in the form of a crystalline solid. In some aspects provided herein, the compound of formula (I) is in the form of a (S)-propylene glycol ((S)-PG) solvate, which has the structure:

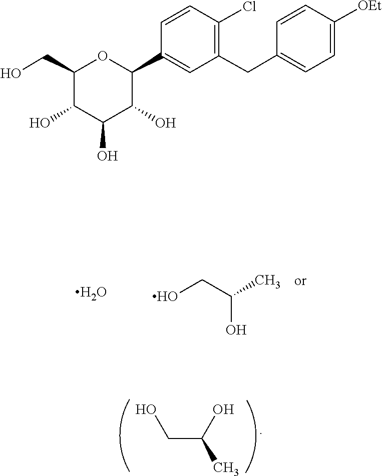

In at least one embodiment, the compound of formula (II) is in the form of a pharmaceutically acceptable solvate, mixed solvate, or complex. In some aspects provided herein, the compound of formula (II) is in the form of a non-crystalline solid. In some aspects provided herein, the compound of formula (II) is in the form of a crystalline solid.

In some aspects provided herein, the pharmaceutical compositions are administered to the patient orally. In some aspects provided herein, the pharmaceutical compositions are administered to the patient in a tablet form.

In some aspects provided herein, the pharmaceutical composition comprising dapagliflozin comprises a dose equivalent of about 2.5 mg/day to about 10 mg/day dapagliflozin is administered to the patient. In some aspects provided herein, the pharmaceutical composition comprising dapagliflozin comprises a dose equivalent of about 2.5 mg/day, about 5 mg/day, or about 10 mg/day dapagliflozin is administered to the patient. In some aspects provided herein, the pharmaceutical composition comprising dapagliflozin comprises a dose equivalent of about 5 mg/day dapagliflozin is administered to the patient once per day. In some aspects provided herein, the pharmaceutical composition comprising dapagliflozin comprises a dose equivalent of about 10 mg/day dapagliflozin is administered to the patient once per day.

In some aspects provided herein, the pharmaceutical composition comprising ambrisentan comprises a dose equivalent of about 2.5 mg/day to about 10 mg/day ambrisentan is administered to the patient. In some aspects provided herein, the pharmaceutical composition comprising ambrisentan comprises a dose equivalent of about 2.5 mg/day, about 5 mg/day, or about 10 mg/day ambrisentan is administered to the patient. In some aspects provided herein, the pharmaceutical composition comprising ambrisentan comprises a dose equivalent of about 5 mg/day ambrisentan is administered to the patient once per day. In some aspects provided herein, the pharmaceutical composition comprising ambrisentan comprises a dose equivalent of about 10 mg/day ambrisentan is administered to the patient once per day.

For patients requiring a mechanical ventilator, the tablet will be crushed and flushed with water down the patient's nastrogastric tube.

In some aspects provided herein, the pharmaceutical compositions are administered for one week. In some aspects provided herein, the pharmaceutical compositions are administered for two weeks. In some aspects provided herein, the pharmaceutical compositions are administered for three weeks. In some aspects provided herein, the pharmaceutical compositions are administered for four weeks. In some aspects provided herein, the pharmaceutical compositions are administered for one day to four weeks. In some aspects provided herein, the pharmaceutical compositions are administered for more than four weeks and up to eight weeks. In some aspects, the pharmaceutical compositions are administered for 30 days.

In some aspects provided herein, the administration increases urinary glucose excretion in the patient. In some aspects provided herein, the administration increases gluconeogenesis in the patient. In some aspects provided herein, the administration increases amino acid utilization in the patient. In some aspects provided herein, the administration decreases fasting alanine levels in the patient. In some aspects provided herein, the administration increases fasting urea levels in the patient. In some aspects provided herein, the administration increases autophagy in the patient. In some aspects provided herein, the administration results in at least one of the following outcomes: extends the length of time to death; extends the length of time to new and/or worsening organ dysfunction; reduces the length of time to hospital discharge; reduces the level of elevated cardiac biomarkers (e.g., troponin, NTproBNP); and/or reduces the level of dyspnea severity.

In some aspects provided herein, the administration results in a reduction of infection in SARS-CoV-2. In some aspects provided herein, the administration results in an improved score as assessed by the National Early Warning Score 2 (NEWS 2). In some aspects provided herein, the administration results in an improved clinical status, as measured on a 7-point ordinal scale at Day 15 or at discharge from hospital, whichever is earlier. In some aspects provided herein, the administration results in an increase in number of days alive, out of the hospital, and not on renal replacement therapy. In some aspects provided herein, the administration results in a greater proportion of patients with acute coronary syndrome, wherein acute coronary syndrome is defined as abnormal troponin level above 99th percentile of the local laboratory reference range or, if abnormal at baseline, further rise in troponin levels accompanied by at least 1 of the following: 1) ischemic symptoms 2) ischemic ST-segment changes on ECG. In some aspects provided herein, the new and/or worsening organ dysfunction is measured by one or more of the following: required intubation; acute renal insufficiency; and/or acute cardiovascular insufficiency.

### DETAILED DESCRIPTION OF THE DISCLOSURE

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. Patients or subjects in need of treatment of treatment can include those diagnosed with coronavirus 2019 (COVID 19) and those who have been infected with severe acute respiratory syndrome coronavirus 2 (SARS-CoV-2).

A “therapeutically effective amount” or “effective amount” refers to an amount of at least one compound of the present disclosure or a pharmaceutical composition comprising at least one such compound that, when administered to a patient, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the patient. Patients may generally be monitored for therapeutic effectiveness using assays suitable for the disease, disorder and/or condition being treated or prevented, which assays will be familiar to those having ordinary skill in the art and are described herein. The level of a compound that is administered to a patient may be monitored by determining the level of the compound (or a metabolite of the compound) in a biological fluid, for example, in the blood, blood fraction (e.g, serum), and/or in the urine, and/or other biological sample from the patient. Any method practiced in the art to detect the compound, or metabolite thereof: may be used to measure the level of the compound during the course of a therapeutic regimen

Alternatively, the pharmacologic and/or physiologic effect may be prophylactic, i.e., the effect completely or partially prevents a disease or symptom thereof. In this respect, the disclosed method comprises administering a “prophylactically effective amount” of a drug (e.g., dapagliflozin or a pharmaceutically acceptable salt, solvate, mixed solvate, complex, or prodrug thereof and ambrisentan or a pharmaceutically acceptable salt, solvate, mixed solvate, complex, or prodrug thereof). A “prophylactically effective amount” refers to an amount effective, at dosages and for periods of time necessary, to achieve a desired prophylactic result (e.g., prevention of SARS-CoV-2 infection or disease onset).

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a cynomolgus monkey. In some aspects, the subject is a human.

The terms “administer,” “administering,” “administration,” and the like, as used herein, refer to methods that may be used to enable delivery of a drug, e.g., dapagliflozin or a pharmaceutically acceptable salt, solvate, mixed solvate, complex, or prodrug thereof and ambrisentan or a pharmaceutically acceptable salt, solvate, mixed solvate, complex, or prodrug thereof, as described herein. Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. In some aspects, the SGLT2 inhibitor and the ET inhibitor are administered orally.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

A “sterile” formulation is aseptic or essentially free from living microorganisms and their spores.

As used herein, the term “prodrug” refers to, for example, esters and carbonates that may be converted, for example, under physiological conditions or by solvolysis, to dapagliflozin or ambrisentan. Thus, the term prodrug includes metabolic precursors of dapagliflozin or ambrisentan that are pharmaceutically acceptable. The term prodrug also includes covalently bonded carriers that release dapagliflozin or ambrisentan in vivo when such prodrug is administered to a patient. Non-limiting examples of prodrugs include esters and carbonates.

Various forms of prodrugs are known in the art. For examples of such prodrug derivatives, see: (1) Design of Prodrugs, edited by H. Bundgaard, (Elsevier, 1985) and Methods in Enzymology, Vol. 42, p. 309-396, edited by K. Widder, et al. (Academic Press, 1985); (2) A Textbook of Drug Design and Development, edited by Krogsgaard-Larsen and H. Bundgaard, Chapter 5 “Design and Application of Prodrugs”, by H. Bundgaard p. 113-191 (1991); (3) H. Bundgaard, Advanced Drug Delivery Reviews, 8, 1-38 (1992); (4) H. Bundgaard, et al., Journal of Pharmaceutical Sciences, 77, 285 (1988); and (5) N. Kakeya, et al., Chem Pharm Bull, 32, 692 (1984).

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

Any compositions or methods provided herein can be combined with one or more of any of the other compositions and methods provided herein.

As provided herein, a combination of the compounds having the structure of formula (I) or prodrugs thereof and compounds having the structure of formula (II) or prodrugs thereof can be used in the methods described herein to treat or prevent COVID-19, SARS-CoV-2 infection, and/or symptoms thereof.

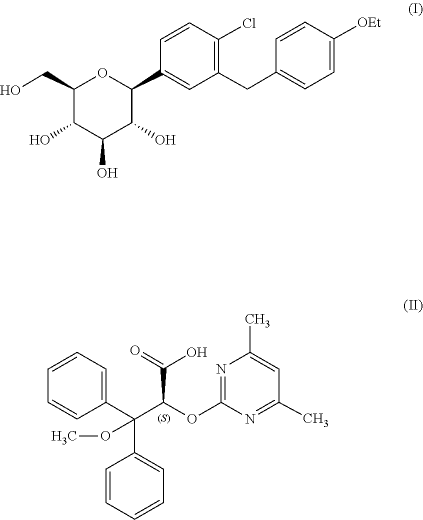

Compounds of formula (I) have been shown to inhibit human renal sodium-dependent glucose transporter 2 (SGLT2), i.e., to be SGLT2 inhibitors (SGLT2i). By inhibiting SGLT2, compounds of formula (I) or prodrugs thereof can reduce reabsorption of filtered glucose and lower the renal threshold for glucose, thereby increasing urinary glucose excretion. Compounds of formula (I) or prodrugs thereof can also reduce sodium reabsorption and increase the delivery of sodium to the distal tube. This may influence several physiological functions including, but not restricted to, lowering both pre- and afterload of the heart and downregulation of sympathetic activity. Compounds of formula (I) and or prodrugs thereof can have a positive impact on both cardiac and renal function.

Compounds of formula (I) have been described, for example, in U.S. Pat. Nos. 6,414,126; 6,515,117, and 7,919,598, each of which is herein incorporated by reference in its entireties.

In at least one embodiment, the compound of formula (I) is D-glucitol, 1,5-anhydro-1-C-[4-chloro-3-[(4-ethoxyphenyl)methyl]phenyl]-,(1S)-compound with (2S)-1,2-propendiol, hydrate (1:1:1), the structure of which is shown below

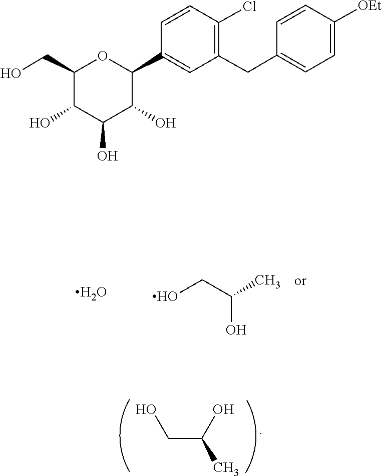

A composition (e.g., a pharmaceutical composition) provided herein can comprise a compound of formula (I) or prodrug thereof. Such a composition can be formulated for oral administration, e.g. in the form of a tablet.

A composition (e.g., a pharmaceutical composition) provided herein can comprise about 2.5 mg of a compound of formula (I) or prodrug thereof. Such a composition can be formulated for oral administration, e.g. in the form of a tablet.

A composition (e.g., a pharmaceutical composition) provided herein can comprise about 5 mg of a compound of formula (I) or prodrug thereof. Such a composition can be formulated for oral administration, e.g. in the form of a tablet.

A composition (e.g., a pharmaceutical composition) provided herein can comprise about 10 mg of a compound of formula (I) or prodrug thereof. Such a composition can be formulated for oral administration, e.g. in the form of a tablet.

A composition (e.g., a pharmaceutical composition) provided herein can comprise a compound of formula (I) or prodrug thereof, wherein the compound is in the form of a crystalline solid or non-crystalline solid.

A composition (e.g., a pharmaceutical composition) provided herein can comprise a compound of formula (I) or prodrug thereof, wherein the compound is in the form of a (S)-propylene glycol ((S)-PG) solvate, which has the structure:

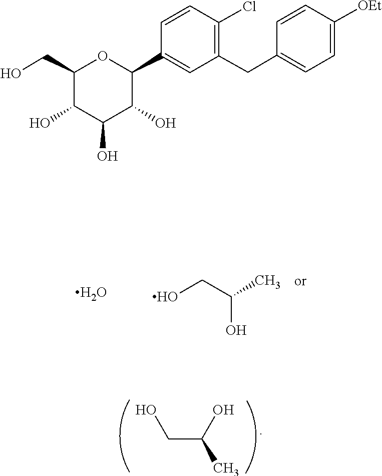

Compounds of formula (II) have been shown to inhibit the endothelin (ET) receptor, i.e, to be ET receptor inhibitors.

Compounds of formula (II) have been described, for example, in U.S. Pat. No. 5,703,017; WO1994025442, each of which is herein incorporated by reference in its entireties.

In at least one embodiment, the compound of formula (II) is (2S)-2-[(4,6-dimethylpyrimidin-2-yl)oxy]-3-methoxy-3,3-diphenylpropanoic acid.

A composition (e.g., a pharmaceutical composition) provided herein can comprise a compound of formula (II) or prodrug thereof. Such a composition can be formulated for oral administration, e.g. in the form of a tablet.

Gattinoni et al (Intensive Care Medicine; DOI: 10.1007/s00134-020-06033-2) also describe the “L” phenotype of COVID-19 related respiratory failure, where there is prominent hypoxia and pulmonary shunting in the absence of marked radiographic changes consistent with ARDS. This may represent an important earlier opportunity to intervene in the progressive respiratory failure which is seen in association with COVID-19 infection. Utilising an endothelin-A (ET-A) receptor antagonist should reduce pulmonary arterial hypertension, pulmonary shunting to hypoxic areas of the lung, and blunt inflammatory activity.

Data from cell culture suggests that interferon-gamma and TNF-alpha, inflammatory markers upregulated in ARDS drive endothelin-1 production. (Scientific Reports, 7:14500; DOI:10.1038/s41598-017-14202-5). Endothelin-1 levels are known to be elevated in patients with ARDS, and reduced clearance of endothelin-1 is also recognised. Persistently elevated endothelin-1 is associated with a worse outcome in patients diagnosed with ARDS. (Am Rev Respir Dis 1993; 148:1169-73). Pre-clinical models of endothelin antagonism in acute lung injury models support this mode of action with respect to intervention in ARDS. Tezosentan, a non-selective endothelin antagonist attenuated lung injury in endotoxaemic sheep and LU-135252, an ET-A selective receptor antagonist improved oxygenation in an experimental pig model of acute lung injury. In a rat airway model of LPS induced lung damage, both mixed and ET-A selective antagonists were shown to reduce microvascular leakage. Interestingly, an endothelin-B (ET-B) selective antagonist showed no effect in that rat model indicating that an ET-A selective antagonist could be beneficial (Critical Care 2005 9:R677; British journal of pharmacology 131 (6), 1129-1134).

Limited data from use of Bosentan (dual ETA/ETB receptor antagonist) in humans supports a positive effect on pulmonary hypertension and refractory hypoxaemia in a patient with ARDS and H7N9 influenza virus infection (Lung 2014 October; 192(5):635-6. doi: 10.1007/s00408-014-9602-9). The patient treated with Bosentan had a rapid and sustained improvement in RVSP which was followed by a gradual improvement in oxygenation

Ambrisentan has greater selectivity for the ET-A receptor compared to bosentan. This has a significant potential advantage in ARDS, maintaining the positive effects of ET-1 at the ET-B receptor including vasodilatation, reduced pulmonary shunting and anti-inflammatory effects and clearance of ET-1 itself which is mediated by the ET-B receptor.

Ambrisentan is administered in conjunction with dapagliflozin both to offset any negative effects of fluid re-distribution in the COVID-19 population associated with ET-A receptor antagonist therapy, and because patients may derive further benefit from dapagliflozin therapy.

As provided herein, a combination of the compounds of formula (I) or prodrugs thereof and compounds of formula (II) or prodrugs thereof can be used in the methods described herein to treat or prevent COVID-19, SARS-CoV-2 infection, and/or symptoms thereof.

The impact of autophagy on viral replication or destruction is complex: some virus families hijack the autophagy/lysosomal process to enhance their replication, while other virus families are actively cleared and destroyed by active autophagy process. The corona virus family appears to be in the latter class, with autophagy lowering the rate replication. Therefore, activated autophagy could be beneficial in controlling the amount and duration of significant viral load and could shorten the course of the disease. Autophagy becomes impaired with age, in individuals with type 2 diabetes and other chronic diseases, and this results in an impaired ability to combat autophagy sensitive viruses.

There is evidence that compounds of formula (I) or prodrugs thereof (e.g., dapagliflozin), by increasing urinary glucose excretion can cause an enhanced overnight fasting state, including increased gluconeogenesis and subsequent amino acid utilization as evidenced by decreased fasting alanine levels and increased fasting urea levels in the blood. It is known that diminished amino acid levels inhibit cellular mammalian target of rapamycin complex 1 (mTORC1 signaling), which activates autophagy and lysosomal function to degrade additional protein to keep gluconeogenesis going. Therefore, the action of that compounds of formula (I) or prodrugs thereof e.g., dapagliflozin) can increase and restore the cellular process of autophagy in individuals with reduced autophagy, including elderly and individuals with chronic diseases such as type 2 diabetes. The process of restored autophagy can aid in the helping remove the virus more rapidly, thereby reducing the risk for serious complications of the infection with lowered morbidity and mortality. In addition, an active autophagy process can also lower the level of inflammation by blunting the impact of cytokines contributing to diminished disease severity. (Wu et al., Int J Biol Sci 12(7):884-897 (2016)).

Furthermore, compounds of formula (I) or prodrugs thereof (e.g., dapagliflozin) have been shown to have a rapid onset effect to reduce the risk for hospitalization for heart failure (HF) and to reduced cardiovascular (CV) death in HF patients with and without type 2 diabetes mellitus. The effect of dapagliflozin on HF is multifactorial, but may be driven by change in blood pressure (BP) and circulating blood volume, lowering both preload and afterload of the heart, causing reduced heart work. Compounds of formula (I) or prodrugs thereof (e.g., dapagliflozin) can protect kidneys from progression to chronic kidney disease in part by reducing intraglomerular pressure.

The lungs of chronically ill patients could be protected from COVID-19 by multiple actions of compounds of formula (I) or prodrugs thereof (e.g., dapagliflozin), including lowering of glucose levels in lung tissue, lowering plasma volume thereby reducing extracellular fluid, and/or enhancing autophagy in critical cells such as endothelial cells and immune cells.

The combined actions of compounds of formula (I) or prodrugs thereof (e.g., dapagliflozin) and compounds of formula (II) or prodrugs thereof (e.g. ambrisentan) can help to combat cytokine storm.

Prophylactic use of a combination of the compounds of formula (I) or prodrugs and compounds of formula (II) or prodrugs thereof in vulnerable individuals may improve cellular and organ health, allowing the individual to combat the virus more effectively.

Night-time fasting, which is permissible in individuals receiving compounds of formula (I) and or prodrugs thereof (e.g., dapagliflozin), can improve treatment or prevention of COVID-19, severe acute respiratory syndrome coronavirus 2 (SARS-CoV-2) infection, and symptoms thereof.

Accordingly, the present disclosure provides methods of treating or preventing coronavirus disease 2019 (COVID-19) in a patient comprising administering to the patient a combination of the a composition comprising an effective amount compound of formula (I) or prodrugs thereof (e.g., dapagliflozin) and a further composition comprising an effective amount compound of formula (II) or prodrugs thereof (e.g. ambrisentan). The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in preparation of a medicament for treating or preventing coronavirus disease 2019 (COVID-19) in a patient. The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in treating or preventing coronavirus disease 2019 (COVID-19) in a patient. The present disclosure also provides uses of a combination of a composition comprising a compound of formula (I) or prodrugs thereof (e.g., dapagliflozin) and a further composition comprising a compound of formula (II) or prodrugs thereof (e.g., ambrisentan) for treating or preventing coronavirus disease 2019 (COVID-19) in a patient.

The present disclosure also provides methods of treating or preventing renal insufficiency induced by severe acute respiratory syndrome coronavirus 2 (SARS-CoV-2) infection in a patient comprising administering to the patient a combination of a composition comprising an effective amount of a compound of formula (I) or prodrugs thereof (e.g., dapagliflozin) and a further composition comprising an effective amount of a compound of formula (II) or prodrugs thereof (e.g., ambrisentan). Renal insufficiency can be evidenced by e.g., a sustained (3 hour) urine production of less than 0.5 ml/kg/hour or introduction of additional diuretic therapy to keep urine production greater than 0.5 ml/kg/hour or doubling of serum creatinine (s-Cr) as compared to baseline. The present disclosure also provides a combination of a composition comprising a compound of formula (I) and prodrugs thereof (e.g., dapagliflozin) and a further composition comprising a compound of formula (II) and prodrugs thereof (e.g., ambrisentan) for use in preparation of a medicament for treating or preventing renal insufficiency induced by SARS-CoV-2 infection in a patient. The present disclosure also provides a combination of a composition comprising a compound of formula (I) or prodrugs thereof (e.g., dapagliflozin) and a further composition comprising a compound of formula (II) and prodrugs thereof (e.g., ambrisentan) for use in treating or preventing renal insufficiency induced by SARS-CoV-2. The present disclosure also provides uses of a combination of a composition comprising a compound of formula (I) or prodrugs thereof (e.g., dapagliflozin) and a further composition comprising a compound of formula (II) and prodrugs thereof (e.g., ambrisentan) for treating or preventing renal insufficiency induced by SARS-CoV-2.

The present disclosure also provides methods of treating or preventing cardiovascular insufficiency induced by SARS-CoV-2 infection in a patient comprising administering to the patient a combination of a composition comprising an effective amount of a compound of formula (I) or prodrugs thereof (e.g., dapagliflozin) and a further composition comprising an effective amount of a compound of formula (I) or prodrugs thereof (e.g., ambrisentan). Cardiovascular insufficiency can be evidenced by, e.g., the need for intravenous vasoactive therapy (e.g., adrenaline, dobutamin, dopamine (≥5 μg/kg(min)), phenylephrine, phosphodiesterase inhibitor, levosimendan, nitroglycerine, noradrenaline, or vasopressin) to keep a mean arterial pressure (MAP) of greater than 70 mm Hg, worsening left ventricular ejection fraction (LVEF), e.g., as confirmed by ultrasonic-echocardiography (UCG), or new diagnosis of acute myocardial infarction (MI). The present disclosure also provides a combination of a composition comprising a compound of formula (I) or prodrugs thereof (e.g., dapagliflozin) and a further composition comprising a compound of formula (II) and prodrugs thereof (e.g., ambrisentan) for use in preparation of a medicament for treating or preventing cardiovascular insufficiency induced by SARS-CoV-2 infection in a patient. The present disclosure also provides a combination of a composition comprising a compound of formula (I) or prodrugs thereof (e.g., dapagliflozin) and a further composition comprising a compound of formula (II) and prodrugs thereof (e.g., ambrisentan) for use in treating or preventing cardiovascular insufficiency induced by SARS-CoV-2 infection in a patient. The present disclosure also provides uses of a combination of a composition comprising a compound of formula (I) or prodrugs thereof (e.g., dapagliflozin) and a further composition comprising a compound of formula (II) and prodrugs thereof (e.g., ambrisentan) for treating or preventing cardiovascular insufficiency induced by SARS-CoV-2 infection in a patient.

The present disclosure also provides methods of treating or preventing respiratory insufficiency induced by SARS-CoV-2 infection in a patient comprising administering to the patient a combination of a composition comprising an effective amount of a compound of formula (I) or prodrugs thereof (e.g., dapagliflozin) and a further composition comprising an effective amount of a compound of formula (II) or prodrugs thereof (e.g., ambrisentan). The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in preparation of a medicament for treating or preventing respiratory insufficiency induced by SARS-CoV-2 infection in a patient. The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in treating or preventing respiratory insufficiency induced by SARS-CoV-2 infection in a patient. The present disclosure also provides uses of a combination of a composition comprising a compound of formula (I) or prodrugs thereof (e.g., dapagliflozin) and a further composition comprising a compound of formula (II) or prodrugs thereof (e.g., ambrisentan) for treating or preventing respiratory insufficiency induced by SARS-CoV-2 infection in a patient.

The present disclosure also provides methods of treating or preventing worsening respiratory insufficiency induced by SARS-CoV-2 infection in a patient comprising administering to the patient a combination of a composition comprising an effective amount of a compound of formula (I) or prodrugs thereof (e.g., dapagliflozin) and a further composition comprising a compound of formula (II) or prodrugs thereof (e.g., ambrisentan). The present disclosure also provides a composition comprising a combination of a compound of formula (I) or prodrugs thereof (e.g., dapagliflozin) and a further composition comprising a compound of formula (II) or prodrugs thereof (e.g., ambrisentan) for use in preparation of a medicament for treating or preventing worsening respiratory insufficiency induced by SARS-CoV-2 infection in a patient. The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in treating or preventing worsening respiratory insufficiency induced by SARS-CoV-2 infection in a patient. The present disclosure also provides uses of a combination of a composition comprising a compound of formula (I) or prodrugs thereof (e.g., dapagliflozin) and a further composition comprising a compound of formula (II) or prodrugs thereof (e.g., ambrisentan) for treating or preventing worsening respiratory insufficiency induced by SARS-CoV-2 infection in a patient.

The present disclosure also provides methods of treating or preventing a cytokine storm in a patient infected by SARS-CoV-2 or a patient with COVID. The methods comprise administering to the patient a composition comprising a combination of an effective amount of a compound of formula (I) or prodrugs thereof (e.g., dapagliflozin) and a further composition comprising an effective amount of a compound of formula (II) or prodrugs thereof (e.g., ambrisentan). The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in preparation of a medicament for treating or preventing a cytokine storm in a patient infected by SARS-CoV-2 or a patient with COVID. The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in treating or preventing a cytokine storm in a patient infected by SARS-CoV-2 or a patient with COVID. The present disclosure also provides uses of a combination of a composition comprising a compound of formula (I) and prodrugs thereof (e.g., dapagliflozin) and a further composition comprising a compound of formula (II) or prodrugs thereof (e.g., ambrisentan) for treating or preventing a cytokine storm in a patient infected by SARS-CoV-2 or a patient with COVID.

The present disclosure also provides methods of reducing the rate of a composite endpoint of death, worsening respiratory insufficiency, or acute insufficiency in renal and/or cardiovascular systems in a patient with COVID-19, comprising administering to the patient a combination of a composition comprising an effective amount of a compound of formula (I) or prodrugs thereof and a further composition comprising an effective amount of a compound of formula (II) or prodrugs thereof (e.g., ambrisentan). The composite endpoint can be reduced relative to an administration regimen where the patient does not receive a pharmaceutical composition comprising an effective amount of a combination of a compound of formula (I) or prodrugs thereof and a further composition comprising an effective amount of a compound of formula (II) or prodrugs thereof. The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in preparation of a medicament for reducing the rate of a composite endpoint of death, worsening respiratory insufficiency, or acute insufficiency in renal and/or cardiovascular systems in a patient with COVID-19. The present disclosure also provides a combination of a composition comprising a compound of formula (I) or prodrugs thereof (e.g., dapagliflozin) and a further composition comprising a compound of formula (II) or prodrugs thereof (e.g., ambrisentan) for use in reducing the rate of a composite endpoint of death, worsening respiratory insufficiency, or acute insufficiency in renal and/or cardiovascular systems in a patient with COVID-19. The present disclosure also provides uses of a combination of a composition comprising a compound of formula (I) or prodrugs thereof (e.g., dapagliflozin) and a further composition comprising a compound of formula (II) or prodrugs thereof (e.g., ambrisentan) for reducing the rate of a composite endpoint of death, worsening respiratory insufficiency, or acute insufficiency in renal and/or cardiovascular systems in a patient with COVID-19.

The present disclosure also provides methods of reducing the rate of a composite endpoint of all-cause mortality or new/worsened organ dysfunction, wherein the new/worsened organ dysfunction is defined as at least one of the following: worsening respiratory insufficiency; new or worsening congestive heart failure (defined as at least one of the following: 1) initiation of new intravenous therapy for heart failure 2) reinstitution of previous intravenous therapy for heart failure 3) increase in current intravenous therapy for heart failure); requirement for vasopressor therapy and/or inotropic or mechanical circulatory support; ventricular tachycardia or fibrillation lasting at least 30 seconds and/or associated with hemodynamic instability or pulseless electrical activity, resuscitated cardiac arrest; and/or initiation of renal replacement therapy.

The present disclosure also provides methods of reducing the rate of a composite endpoint of time to death for any cause; time to new/worsened organ dysfunction; clinical status at day 30 for patients still hospitalized and without any worsening organ dysfunction (using points 3 to 5 of a 7-point ordinal scale); and/or time to hospital discharge.

In some aspects of the methods, the clinical status at day 30 for patients still hospitalized can be assessed via a 7-point scale:

1) Not hospitalized, no limitations on activities

2) Not hospitalized, limitation on activities

3) Hospitalized, not requiring supplemental oxygen

4) Hospitalized, requiring supplemental oxygen

5) Hospitalized, on high flow oxygen devices

6) Hospitalized, on invasive mechanical ventilation or ECMO

7) Death

In some aspects of the methods, new/worsened organ dysfunction can be defined as at least one of the following: respiratory decompensation requiring initiation of invasive or non-invasive mechanical ventilation or continuous positive airway pressure (CPAP) treatment, and/or initiation of veno-venous extracorporeal membrane oxygenation (ECMO); new or worsening congestive heart failure during current hospitalization; requirement for vasopressor therapy and/or inotropic or mechanical circulatory support; ventricular tachycardia or fibrillation lasting at least 30 seconds and/or associated with hemodynamic instability or pulseless electrical activity, or resuscitated cardiac arrest; and/or initiation of renal replacement therapy. In some aspects, new/worsened organ dysfunction can be defined as respiratory decompensation requiring endotracheal intubation and initiation of invasive mechanical ventilation treatment, and/or initiation of veno-venous extracorporeal membrane oxygenation (ECMO).

In some aspects of the methods, compounds for use, and uses provided herein, the compositions comprising a compound of formula (I) or prodrugs thereof (e.g., dapagliflozin) and comprising a compound of formula (II) or prodrugs thereof (e.g., ambrisentan) are administered orally to the patient, sequentially or in the same composition. In some aspects of the methods, compounds for use, and uses provided herein, the compositions comprising a compound of formula (I) or prodrugs thereof (e.g., dapagliflozin) and comprising a compound of formula (II) or prodrugs thereof (e.g., ambrisentan) are administered orally to the patient in the form of tablets, either as discrete tablets or tablets comprising both compounds of formula (I) and formula (II).

In some aspects of the methods, compounds for use, and uses provided herein, the composition comprising a compound of formula (I) or prodrugs thereof (e.g., dapagliflozin) is administered at a dose of 2.5 mg, e.g., once per day. In some aspects of the methods, compounds for use, and uses provided herein, the composition comprising a compound of formula (I) or prodrugs thereof (e.g., dapagliflozin) is administered at a dose of 5 mg, e.g., once per day. In some aspects of the methods, compounds for use, and uses provided herein, the composition comprising a compound of formula (I) or prodrugs thereof (e.g., dapagliflozin) is administered at a dose of 10 mg, e.g., once per day. The dose of 2.5 mg per day, 5 mg per day, or 10 mg per day can be administered orally, e.g., in the form of a tablet.

In some aspects of the methods, compounds for use, and uses provided herein, the composition comprising a compound of formula (II) or prodrugs thereof (e.g., ambrisentan) is administered at a dose of 2.5 mg, e.g., once per day. In some aspects of the methods, compounds for use, and uses provided herein, the composition comprising a compound of formula (II) or prodrugs thereof (e.g., ambrisentan) is administered at a dose of 5 mg, e.g., once per day. In some aspects of the methods, compounds for use, and uses provided herein, the composition comprising a compound of formula (II) or prodrugs thereof (e.g., ambrisentan) is administered at a dose of 10 mg, e.g., once per day. The dose of 2.5 mg per day, 5 mg per day, or 10 mg per day can be administered orally, e.g., in the form of a tablet.

In some aspects of the methods, compounds for use, and uses provided herein, the composition comprising a compound of formula (I) or prodrugs thereof (e.g., dapagliflozin) is administered at a dose of 10 mg, e.g., once per day and is co-administered with a composition comprising a compound of formula (II) or prodrugs thereof (e.g., ambrisentan) is administered at a dose of 2.5 mg, e.g., once per day. In some aspects of the methods, compounds for use, and uses provided herein, the composition comprising a compound of formula (I) or prodrugs thereof (e.g., dapagliflozin) is administered at a dose of 10 mg, e.g., once per day and is co-administered with a composition comprising a compound of formula (II) or prodrugs thereof (e.g., ambrisentan) is administered at a dose of 5 mg, e.g., once per day.

In some aspects of the methods, compounds for use, and uses provided herein, the composition comprising a compound of formula (I) or prodrugs thereof (e.g., dapagliflozin) and a compound of formula (II) or prodrugs thereof (e.g., ambrisentan) is administered in a single tablet.

In some aspects of the methods, compounds for use, and uses provided herein, the composition comprising a compound of formula (I) or prodrugs thereof (e.g., dapagliflozin) and the composition comprising a compound of formula (II) or prodrugs thereof (e.g., ambrisentan) are administered (e.g., daily) for one to four weeks. In some aspects of the methods, compounds for use, and uses provided herein, the composition comprising a compound of formula (I) or prodrugs thereof (e.g., dapagliflozin) and the composition comprising a compound of formula (II) or prodrugs thereof (e.g., ambrisentan) are administered (e.g., daily) for two to four weeks. In some aspects of the methods, compounds for use, and uses provided herein, the composition comprising a compound of formula (I) or prodrugs thereof (e.g., dapagliflozin) and the composition comprising a compound of formula (II) or prodrugs thereof (e.g., ambrisentan) are administered (e.g., daily) for three to four weeks. In some aspects of the methods, compounds for use, and uses provided herein, the composition comprising a compound of formula (I) or prodrugs thereof (e.g., dapagliflozin) and the composition comprising a compound of formula (II) or prodrugs thereof (e.g., ambrisentan) are administered (e.g., daily) for at least four weeks. In some embodiments, provided herein, the pharmaceutical composition and the composition comprising a compound of formula (II) or prodrugs thereof (e.g., ambrisentan) are administered for more than four weeks and up to eight weeks.

In some aspects of the methods, compounds for use, and uses provided herein, the combination of a composition comprising a compound of formula (I) or prodrugs thereof (e.g., dapagliflozin) and the composition comprising a compound of formula (II) or prodrugs thereof (e.g., ambrisentan) are administered (e.g., daily) for 1 week to 60 days. In some aspects of the methods, compounds for use, and uses provided herein, the combination of a composition comprising a compound of formula (I) or prodrugs thereof (e.g., dapagliflozin) and the composition comprising a compound of formula (II) or prodrugs thereof (e.g., ambrisentan) are administered (e.g., daily) for 7 days to 14 days. In some aspects of the methods, compounds for use, and uses provided herein, the combination of a composition comprising a compound of formula (I) or prodrugs thereof (e.g., dapagliflozin) and the composition comprising a compound of formula (II) or prodrugs thereof (e.g., ambrisentan) are administered (e.g., daily) for at least 30 days. In some aspects of the methods, compounds for use, and uses provided herein, the combination of a composition comprising a compound of formula (I) or prodrugs thereof (e.g., dapagliflozin) and the composition comprising a compound of formula (II) or prodrugs thereof (e.g., ambrisentan) are administered (e.g., daily) for 30 days to 60 days. In some aspects of the methods, compounds for use, and uses provided herein, the combination of a composition comprising a compound of formula (I) or prodrugs thereof (e.g., dapagliflozin) and the composition comprising a compound of formula (II) or prodrugs thereof (e.g., ambrisentan) are administered (e.g., daily) for 45 days to 60 days. In some aspects of the methods, compounds for use, and uses provided herein, the combination of a composition comprising a compound of formula (I) or prodrugs thereof (e.g., dapagliflozin) and the composition comprising a compound of formula (II) or prodrugs thereof (e.g., ambrisentan) are administered (e.g., daily) for at least 60 days.

In some aspects of the methods, compounds for use, and uses provided herein, the administration of a combination of the composition comprising a compound of formula (I) or prodrugs thereof (e.g., dapagliflozin) and the composition comprising a compound of formula (II) or prodrugs thereof (e.g., ambrisentan) decreases the incidence of or the likelihood of renal insufficiency in the patient. The decrease can occur, e.g., within 30 days.

In some aspects of the methods, compounds for use, and uses provided herein, the administration of a combination of the composition comprising a compound of formula (I) or prodrugs thereof (e.g., dapagliflozin) and the composition comprising a compound of formula (II) or prodrugs thereof (e.g., ambrisentan) decreases the incidence of or the likelihood of cardiovascular insufficiency in the patient. The decrease can occur, e.g., with 30 days.

In some aspects of the methods, compounds for use, and uses provided herein, the administration of a combination of the composition comprising a compound of formula (I) or prodrugs thereof (e.g., dapagliflozin) and the composition comprising a compound of formula (II) or prodrugs thereof (e.g., ambrisentan) decreases the incidence of or the likelihood of respiratory insufficiency in the patient. The decrease can occur, e.g., with 30 days.

In some aspects of the methods, compounds for use, and uses provided herein, the administration of a combination of the composition comprising a compound of formula (I) or prodrugs thereof (e.g., dapagliflozin) and the composition comprising a compound of formula (II) or prodrugs thereof (e.g., ambrisentan) decreases the incidence of or the likelihood of worsening respiratory insufficiency in the patient. The decrease can occur, e.g., with 30 days.

In some aspects of the methods, compounds for use, and uses provided herein, the administration of a combination of the composition comprising a compound of formula (I) or prodrugs thereof (e.g., dapagliflozin) and the composition comprising a compound of formula (II) or prodrugs thereof (e.g., ambrisentan) increases the time to (delays) renal insufficiency in the patient.

In some aspects of the methods, compounds for use, and uses provided herein, the administration of a combination of the composition comprising a compound of formula (I) or prodrugs thereof (e.g., dapagliflozin) and the composition comprising a compound of formula (II) or prodrugs thereof (e.g., ambrisentan) increases the time to (delays) cardiovascular insufficiency in the patient.

In some aspects of the methods, compounds for use, and uses provided herein, the administration of a combination of the composition comprising compound of formula (I) or prodrugs thereof (e.g., dapagliflozin) and the composition comprising a compound of formula (II) or prodrugs thereof (e.g., ambrisentan) increases the time to (delays) respiratory insufficiency in the patient.

In some aspects of the methods, compounds for use, and uses provided herein, the administration of a combination of the composition comprising compound of formula (I) or prodrugs thereof (e.g., dapagliflozin) and the composition comprising a compound of formula (II) or prodrugs thereof (e.g., ambrisentan) increases the time to (delays) worsening respiratory insufficiency in the patient.

In some aspects, the administration of a combination of the composition comprising compound of formula (I) or prodrugs thereof (e.g., dapagliflozin) and the composition comprising a compound of formula (II) or prodrugs thereof (e.g., ambrisentan) decreases hemoglobin A1c (HbA1C) in the patient. In some aspects, the administration of a combination of the composition comprising compound of formula (I) or prodrugs thereof (e.g., dapagliflozin) and the composition comprising a compound of formula (II) or prodrugs thereof (e.g., ambrisentan) decreases systolic blood pressure in the patient.

In some aspects of the methods, compounds for use, and uses provided herein, a patient has confirmed or suspected SARS-CoV-2 infection requiring hospitalization.

In some aspects of the methods, compounds for use, and uses provided herein, a patient has a history of any one or more of the following conditions: Heart Failure, including HFrEF and HFpEF; Myocardial infarction, including acute and chronic; Coronary artery disease; Stroke, including ischemic, hemorrhagic, or undetermined; Hypertension; Chronic kidney disease; and/or Type 2 diabetes.

In some aspects of the methods, compounds for use, and uses provided herein, a patient has respiratory insufficiency. In some aspects, a patient has an O₂ saturation of ≤94% without additional oxygen.

In some aspects of the methods, compounds for use, and uses provided herein, a patient has pneumonia. The pneumonia can be pneumonia that has been confirmed by chest imaging.

In some aspects of the methods, compounds for use, and uses provided herein, a patient is a patient that does not have insufficiency in any organ system other than renal and/or cardiovascular organ systems.

In some aspects of the methods, compounds for use, and uses provided herein, a patient is not mechanically ventilated. In some aspects of the methods provided herein, a patient not mechanically ventilated via endotracheal intubation.

In some aspects of the methods, compounds for use, and uses provided herein, a patient does not have type 2 diabetes mellitus (T2DM.) In some aspects of the methods, compounds for use, and uses provided herein, a patient has T2DM.

In some aspects of the methods, compounds for use, and uses provided herein, a patient is a human patient. In some aspects the patient is at least 40 years old. In some aspects, the patient is at least 50 years old. In some aspects, the patient is at least 60 years old. In some aspects, the patient is at least 70 years old. In some aspects, the patient is at least 80 years old. In some aspects, the patient is at least 90 years old.

The following example further illustrates the disclosure but, of course, should not be construed as in any way limiting its scope.

### Example 1: Dapagliflozin and Ambrisentan for Treatment of COVID-19

A study of the effect of dapagliflozin and ambrisentan on the modulation of abnormal pulmonary vascular response to COVID-19 infection and protection against end-organ damage is conducted.

### Patients

Retrospective data from the COVID19 outbreak in Wuhan, China have revealed divergence of a range of biomarkers, in the early and mid-phases of infection, which discriminate between survivors and non-survivors (Zhou et al 2020, DOI:10,21203/rs.3.rs-18079/v1). Several of these (D-dimer, neutrophil:lymphocyte Ratio (NLR), ferritin, LDH), diverge early in the clinical course, and IL6 later). Data from the first 200 patients admitted to King's College Hospital (Sneep et al. under review) have been used to inform patient selection. Clinical and laboratory data, were modelled using penalised (LASSO) logistic regression to select variables with the most prognostic value. The following variables were considered: Age, gender, non-white ethnicity, radiographic severity on chest radiograph, diabetes, hypertension, neutrophils, lymphocytes, CRP. The radiological severity score was calculated using the method described by Wong et al, 2020 (DOI:org/10.1148/radiol.20200201160). A score of 0-4 was assigned to each lung depending on the extent of involvement by consolidation or ground glass opacities. 0=no involvement, 1=<25%, 2=25-49%, 3=50-75%, 4=>75% involvement. The scores for each lung were summed to produce a final severity score ranging from 0-8. Radiographs were scored by two emergency department clinicians after a brief training. Interrater reliability was 90.5%.

The outcome modelled was either admission to ICU or death during follow up. The variables selected from the LASSO model were: radiographic severity, male gender, CRP, non-white ethnicity, diabetes, hypertension, and neutrophils (AUC=0.86). Age was also selected since It was predictive in a non-linear manner (attributable to a much lower rate of ICU admission with older patients) with an inflection point around 50 years of age. For CRP and neutrophils the association was linear on the logarithmic scale with no clear threshold effect. For simplicity and pragmatic reasons, cut points were selected for continuous variables at points where risk of poor outcome was considered clinically important. From all the variables, radiographic severity was by far the strongest predictor of progression.

A risk count was calculated by summing (i.e. patients receive 1 point for) each of the following features on admission:
Radiographic severity score>3
Male gender
Non-white ethnicity
Diabetes
Hypertension
Neutrophils>8.0
Age>40
CRP>40

The corresponding risks of ICU admission or death in the KCH sample associated with this score are shown in the figure below. All admissions presented with at least one of these risk factors (mean 4.4). 83% scored 3 or above (probability of admission to ICU or death=13%), 65% scored 4 or above (probability=21%).

Based upon these data, and the importance of radiographic severity, we selected a score of 3 if radiographic severity score is met, or 4 or higher otherwise as a threshold that captures patients with sufficient risk of progression to justify the risks of immune modulation. This accounted for 71% of the KCH sample. Specifically, individuals meeting this criterion had a 39% risk of admission to ICU or death, versus 9% in those that did not (odds ratio=5.9).

To be included in the trial the participant must:
be aged 18 and over
have clinical picture strongly suggestive of COVID-19-related disease (with/without positive COVID-19 test) AND
   Risk count (as defined above)>3 or
   ≥3 if risk count includes “Radiographic severity score>3”.

Patients do not participate if they meet any of the following exclusion criteria:

The presence of any of the following will preclude participant inclusion:
1. Inability to supply direct informed consent from patient or from Next of Kin or Independent Healthcare Provider on behalf of patient
2. Mechanical ventilation at time of screening
3. Contraindications to study drugs, including hypersensitivity to the active substances or any of the excipients
4. Currently on any of the study investigational medicinal products
5. Known active tuberculosis (no blood screening required)
6. Known active Hepatitis B or C (no blood screening required); active varicella zoster.
7. Concurrent participation in an interventional clinical trial (observational studies allowed)
8. Patient moribund at presentation or screening
9. Pregnancy at screening
10. Unwilling to stop breastfeeding during treatment period
11. Alanine transaminase/aspartate transaminase (ALT/AST)>5 times the upper limit of normal
12. Stage 4 severe chronic kidney disease or requiring dialysis (i.e. Cockcroft Gault estimated glomerular filtration rate (creatinine clearance)<30 ml/min/1.73 m\hat{ }2)
13. Any medical history or clinically relevant abnormality that is deemed by the principal investigator and/or medical monitor to make the patient ineligible for inclusion because of a safety concern.
14. Type 1 diabetes
15. Previous hospital admission with ketoacidosis
16. History of symptomatic heart failure within 3 months of admission
17. Blood pressure below 110/80 mmHg at admission
18. Raised anion gap metabolic acidosis and/or measured blood ketones>3 mmol/l at screening as defined by pH<7.25 and blood ketones>3.0 mmol/l.

Study Procedures: Approximately 460 patients will be screened and randomized on Day 0. Patients receive either 10 mg dapagliflozin and 5 mg ambrisentan or placebo for 14 days, once daily.

Results: Primary Objective—To determine whether dapagliflozin 10 mg and ambrisentan 5 mg reduces the composite of progression of patients with COVID-19-related disease to organ failure or death in patients hospitalized with COVID-19. The primary objective will be measured as time to incidence (up to Day 14) of the composite endpoint of: first occurrence of either death from any cause or new/worsened organ dysfunction through 30 days of follow up, defined as at least one of the following: respiratory decompensation requiring initiation of invasive or non-invasive mechanical ventilation or continuous positive airway pressure (CPAP) treatment, and/or initiation of veno-venous extracorporeal membrane oxygenation (ECMO); new or worsening congestive heart failure during current hospitalization; requirement for vasopressor therapy and/or inotropic or mechanical circulatory support; ventricular tachycardia or fibrillation lasting at least 30 seconds and/or associated with hemodynamic instability or pulseless electrical activity, or resuscitated cardiac arrest; and/or initiation of renal replacement therapy.

Renal insufficiency is defined as sustained (3 hour) urine production of less than 0.5 ml/kg/hour or introduction of additional diuretic therapy to keep urine production greater than 0.5 ml/kg/hour or doubling of serum creatinine (s-Cr) as compared to baseline.

Cardiovascular insufficiency is defined as the need for intravenous vasoactive therapy (e.g., adrenaline, dobutamin, dopamine (≥5 μg/kg(min)), phenylephrine, phosphodiesterase inhibitor, levosimendan, nitroglycerine, noradrenaline, or vasopressin) to keep a mean arterial pressure (MAP) of greater than 70 mmHg, worsening left ventricular ejection fraction (LVEF) as confirmed by ultrasonic-echocardiography (UCG), or new diagnosis of acute myocardial infarction (MI).

Congestive heart failure is defined as at least one of the following 1) initiation of new intravenous therapy for heart failure 2) reinstitution of previous intravenous therapy for heart failure 3) increase in current intravenous therapy for heart failure.

A composite outcome measure will be assessed to evaluate:

### Primary Outcome Measure:

Time to incidence (up to Day 14) of the composite endpoint of:
Death
Mechanical ventilation
ECMO
Cardiovascular organ support (balloon pump or inotropes)
Renal failure (by Cockcroft-Gault formula)<15 ml/min/1.73 m\hat{ }2), haemofiltration or dialysis

### Secondary Outcome Measure:

Change in clinical status as assessed on 7-point ordinal scale compared to baseline
Time to each of the individual endpoints of the composite primary outcome measure
Proportion of patients with adverse events of special interest in each arm
Time to Sp0₂>94% on room air (excluding chronically hypoxic individuals)
Time to first negative SARS-CoV2 PCR
Duration of oxygen therapy (days)
Duration of hospitalisation (days)
All cause mortality at day 28
Time to clinical improvement (defined as ≥2 point improvement from day 1 on 7-point ordinal scale)

The following additional objectives will be evaluated:
Changes in biochemical predictors and immunoinflammatory signatures of therapeutic response and to inform algorithm development
Clinical and biomarker predictors of therapeutic response
Biomarkers of pharmacodynamic response

Dapagliflozin and ambrisentan or placebo will be administered for 7 days, with the option to extend to 14 days, with follow-up assessments every 24 hours until either Day 14, hospital discharge, or death.

Subjects will continue to be enrolled in a treatment arm until one or more of the following have been demonstrated: 1) Efficacy, 2) Futility, 3) Toxicity.

The proportions in the placebo group and the dapagliflozin/ambrisentan group are compared to demonstrate that dapagliflozin/ambrisentan decreases the occurrence of renal insufficiency, cardiovascular insufficiency, worsened respiratory insufficiency, and/or death.

The times to occurrence of renal insufficiency, cardiovascular insufficiency, worsened respiratory insufficiency, and/or death in the placebo group and the dapagliflozin/ambrisentan group are compared to demonstrate that dapagliflozin/ambrisentan delays renal insufficiency, cardiovascular insufficiency, worsened respiratory insufficiency, and/or death.

The total number of days alive, out of hospital, and/or free from mechanical ventilation in the placebo group and the dapagliflozin/ambrisentan group are compared to demonstrate that dapagliflozin/ambrisentan increases total number of days alive, out of hospital, and/or free from mechanical ventilation.

The total number of days alive, not in the ICU, and/or free from mechanical ventilation in the placebo group and the dapagliflozin/ambrisentan group are compared to demonstrate that dapagliflozin/ambrisentan increases total number of days alive, not in the ICU, and/or free from mechanical ventilation.

Safety is also assessed in the placebo group and the dapagliflozin/ambrisentan group to confirm that dapagliflozin/ambrisentan is safe, including assessment of serious adverse events, acute kidney injury (defined as doubling of s-Creatinine compared to baseline), and incidence of diabetic ketoacidosis.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Various aspects of this disclosure are described herein, including the best mode known to the inventors for carrying out the disclosure. Variations of those aspects may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of treating and/or preventing coronavirus disease 2019 (COVID-19) in a patient comprising administering to the patient a combination of a pharmaceutical composition comprising an effective amount of a compound of formula (I) 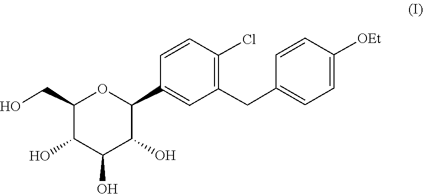 [SMILES: CCOc1ccc(Cc2cc([C@@H]3O[C@H](CO)[C@@H](O)[C@H](O)[C@H]3O)ccc2Cl)cc1]
or prodrugs thereof and a pharmaceutical composition comprising an effective amount of a compound of formula (II) 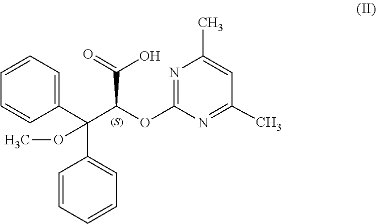
or prodrugs thereof.

2. A method of treating or preventing renal insufficiency induced by severe acute respiratory syndrome coronavirus 2 (SARS-CoV-2) infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

3. A method of treating or preventing cardiovascular insufficiency induced by SARS-CoV-2 infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

4. A method of treating or preventing respiratory insufficiency induced by SARS-CoV-2 infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

5. A method of treating or preventing worsening respiratory insufficiency induced by SARS-CoV-2 infection in a patient comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

6. A method of treating and/or preventing a cytokine storm in a patient infected by SARS-CoV-2 or a patient with COVID-19, the method comprising administering to the patient a combination of a pharmaceutical composition comprising an effective amount of a compound of formula (I) or prodrugs thereof and a pharmaceutical composition comprising an effective amount of a compound of formula (II) or prodrugs thereof.

7. A method of reducing the rate of a composite endpoint of death, worsening respiratory insufficiency, or acute insufficiency in renal and/or cardiovascular systems in a patient with COVID-19, comprising administering to the patient a combination of a pharmaceutical composition comprising an effective amount of a compound of formula (I) or prodrugs thereof and a pharmaceutical composition comprising an effective amount of a compound of formula (II) or prodrugs thereof, wherein the rate of the composite endpoint is reduced relative to an administration regimen where the patient does not receive a combination of a pharmaceutical composition comprising an effective amount of a compound of formula (I) or prodrugs thereof and a pharmaceutical composition comprising an effective amount of a compound of formula (II) or prodrugs thereof.

8. The method of any one of claims 1-7, wherein the patient has confirmed or suspected SARS-CoV-2 infection requiring hospitalization.

9. The method of claims 1-8, wherein the patient has a history of any one or more of the following conditions:
a. heart failure, including heart failure with reduced ejection fraction (HFrEF) and heart failure with preserved ejection fraction (HFpEF);
b. myocardial infarction, including acute and chronic;
c. coronary artery disease;
d. stroke, including ischemic, hemorrhagic, or undetermined;
e. hypertension;
f. chronic kidney disease; and/or
g. Type 2 diabetes.

10. The method of any one of claims 1-9, wherein the patient has respiratory insufficiency.

11. The method of any one of claims 1-9, wherein the patient has an O₂ saturation of ≥94% with low-flow supplemental oxygen.

12. The method of any one of claims 1-11, wherein the patient has pneumonia.

13. The method of any one of claims 1-12, wherein the patient is not mechanically ventilated via endotracheal intubation.

14. The method of any one of claims 1-13, wherein the patient does not have insufficiency in an organ system other than the renal and cardiovascular organ systems.

15. The method of any one of claims 1-14, wherein the patient does not have type 2 diabetes mellitus (T2DM.)

16. The method of claim 15, wherein the patient is at least 40 years of age, at least 50 years of age, at least 60 years of age, at least 70 years of age, at least 80 years old, or at least 90 years old.

17. The method of any one of claims 1-16, wherein the compound of formula (I) is in the form of a pharmaceutically acceptable solvate, mixed solvate, or complex.

18. The method of any one of claims 1-17, wherein the pharmaceutical compositions are administered to the patient orally.

19. The method of any one of claims 1-18, wherein the pharmaceutical compositions are administered to the patient in a tablet form.

20. The method of any one of claims 1-19, wherein the pharmaceutical composition comprising dapagliflozin comprises a dose equivalent of about 2.5 mg/day to about 10 mg/day dapagliflozin and wherein the pharmaceutical composition comprising ambrisentan comprises a dose equivalent of about 2.5 mg/day to about 10 mg/day ambrisentan is administered to the patient.

21. The method of claim 20, wherein the pharmaceutical composition comprising dapagliflozin comprises a dose equivalent of about 2.5 mg/day, about 5 mg/day, or about 10 mg/day dapagliflozin and wherein the pharmaceutical composition comprising ambrisentan comprises a dose equivalent of about 2.5 mg/day, about 5 mg/day, or about 10 mg/day ambrisentan is administered to the patient.

22. The method of any one of claims 1-21, wherein the pharmaceutical composition comprising dapagliflozin comprises a dose equivalent of about 5 mg/day dapagliflozin and wherein the pharmaceutical composition comprising ambrisentan comprises a dose equivalent of about 5 mg/day ambrisentan is administered to the patient once per day.

23. The method of any one of claims 1-21, wherein the pharmaceutical composition comprising dapagliflozin comprises a dose equivalent of about 10 mg/day dapagliflozin and wherein the pharmaceutical composition comprising ambrisentan comprises a dose equivalent of about 5 mg/day ambrisentan is administered to the patient once per day.

24. The method of any one of claims 1-21, wherein the pharmaceutical composition comprising dapagliflozin comprises a dose equivalent of about 10 mg/day dapagliflozin and wherein the pharmaceutical composition comprising ambrisentan comprises a dose equivalent of about 10 mg/day ambrisentan is administered to the patient once per day.

25. The method of any one of claims 1-24, wherein the pharmaceutical composition is administered for one week.

26. The method of claim 25, wherein the pharmaceutical composition is administered for two weeks.

27. The method of claim 26, wherein the pharmaceutical composition is administered for three weeks.

28. The method of claim 27, wherein the pharmaceutical composition is administered for four weeks.

29. The method of any one of claims 1-25, wherein the pharmaceutical composition is administered for one day to four weeks.

30. The method of any one of claims 1-29, wherein the administration increases urinary glucose excretion in the patient.

31. The method of any one of claims 1-30, wherein the administration increases gluconeogenesis in the patient.

32. The method of any one of claims 1-31, wherein the administration increases amino acid utilization in the patient.

33. The method of any one of claims 1-32, wherein the administration decreases fasting alanine levels in the patient.

34. The method of any one of claims 1-33, wherein the administration increases fasting urea levels in the patient.

35. The method of any one of claims 1-34, wherein the administration increases autophagy in the patient.

36. The method of any one of claims 1-35, wherein the administration results in at least one of the following outcomes:
a. extends the length of time to death;
b. extends the length of time to new and/or worsening organ dysfunction;
c. reduces the length of time to hospital discharge;
d. reduces the level of elevated cardiac biomarkers (troponin, NTproBNP); and/or
e. reduces the level of dyspnea severity.

37. The method of claim 36, wherein the new and/or worsening organ dysfunction is measured by one or more of the following:
a. required intubation;
b. acute renal insufficiency; and/or
c. acute cardiovascular insufficiency.
